(19) United States
(12) Patent Application Publication
(10) Pub. No.: US 2003/0229105 A1
(43) Pub. Date: Dec. 11, 2003

(54) Treatment of autoimmune disorders

(75) Inventor: Fatah Kashanchi, Washington, DC (US)

Correspondence Address: LAHIVE & COCKFIELD, 28 STATE STREET, BOSTON, MA, 02109, US

(73) Assignee: Cyclacel Limited, London (GB)

(21) Appl. No.: 10/153,441

(22) Filed: May 21, 2002

(51) Int. Cl. A61K 31/52

(52) U.S. Cl. 514/263.2; 514/263.22; 514/263.4

(57) ABSTRACT

The present invention relates to the use of a compound of formula I

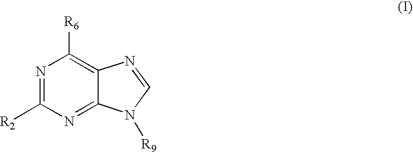

wherein

R₂ is R—NH wherein R is a branched or unbranched alkyl radical, a piperidinyl group or pyrrolidinyl group, each of which may be optionally substituted by one or more —OH, halogen, amino or hydroxyalkyl groups;

R₆ is phenylamino, benzylamino or pyridyl-methylamino, indan-5-amino, where in each case the aryl group may be unsubstituted or substituted by one or more —OR″, halogen, NO₂, amino or COOR′ groups, wherein R′ and R″ are each independently H or a branched or unbranched alkyl group; and

R₉ is a branched or unbranched alkyl group or a cycloalkyl group;

in the treatment of an autoimmune disorder.

The invention further relates to a method of treating an autoimmune disorder comprising administering to a subject in need thereof a therapeutically effective amount of a compound of formula I.

### BACKGROUND

The present invention relates to compounds that are useful in the treatment of autoimmune disorders. More specifically, the invention relates to compounds that have applications in the treatment of HIV-1-related disorders.

Human immunodeficiency virus type 1 (HIV-1) is the etiologic agent of AIDS (3, 11). The HIV-1 infection life cycle can be divided into pre- and postintegration phases, and successful HIV-1 infection is closely related to the host cell cycle progression (33). HIV-1 can infect both dividing and quiescent cells; such as the nondividing T lymphocytes (42, 44), terminally differentiated macrophages (48), brain microglial cells (46, 26), and cells that are artificially arrested in the G₁/S or G₂ phases of the cell cycle (26, 43, 25, 27). However, productive viral infection of HIV-1 is restricted only to dividing cells (49, 5). The preintegration stage of HIV-1 infection can be restricted at either reverse transcription (49) or integration levels (5). The postintegration restriction of HIV-1 transcription is mainly regulated by cellular transcription factors (41) and enzymatic activities of cellular proteins, such as cdk9/cyclin T (20, 51, 47, 13, 21) and cdk7/cyclin H (8, 33, 50, 34), which play a critical role in Tat-mediated transactivation.

Reciprocally, HIV-1 has evolved various means to perturb the cell cycle to optimize the cellular conditions in favor of its own replication. Previous studies have indicated that HIV-1 encoded viral protein R (Vpr) can arrest the cell cycle at the G₂ phase transiently by retaining the G₂/M p34cdc2 in the tyrosine phosphorylated inactive state (18, 14, 19). Blocking the cell cycle at the G₂ phase prolongs the active promoter stage, allowing optimal HIV-1 transcription (18).

The present invention seeks to provide compounds that are useful in the treatment of autoimmune disorders, especially HIV-1-related disorders. In particular, the compounds of interest include, and are derived from, purine-based cdk inhibitors.

To date, there has been no teaching or suggestion in the prior art that purine-based cdk inhibitors would be suitable for the treatment of autoimmune disorders, and in particular, HIV-1-related disorders.

### STATEMENT OF INVENTION

In a first aspect, the invention relates to the use of a compound of formula I

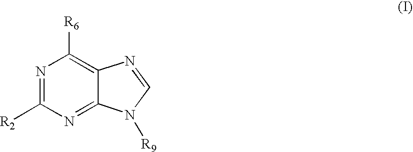

wherein

R₂ is R—NH wherein R is a branched or unbranched alkyl radical, a piperidinyl group or pyrrolidinyl group, each of which may be optionally substituted by one or more —OH, halogen, amino or hydroxyalkyl groups;

R₆ is phenylamino, benzylamino or pyridyl-methylamino, indan-5-amino, where in each case the aryl group may be unsubstituted or substituted by one or more —OR″, halogen, NO₂, amino or COOR′ groups, wherein R′ and R″ are each independently H or a branched or unbranched alkyl group; and

R₉ is a branched or unbranched alkyl group or a cycloalkyl group;

in the treatment of an autoimmune disorder.

A second aspect of the invention relates to a method of treating an autoimmune disorder comprising administering to a subject in need thereof a therapeutically effective amount of a compound of formula I.

More specifically, the invention provides evidence that cdk specific inhibitors are effective drugs that inhibit HIV-1 replication. The inhibition has been observed in HIV-1 latently infected monocytes and T cells, which is associated with the inhibition of viral transcription. Similar results were also obtained in infected activated peripheral blood mononuclear cells (PBMC) with either primary syncytium-inducing (SI) or non-syncytium-inducing (NSI) HIV-1 isolates. Roscovitine inhibited cdk2, -7, and -9 kinase activity with similar 50% inhibitory concentrations (IC₅₀ₛ). In addition, Roscovitine could selectively induce apoptosis in HIV-1-infected cells, as made apparent by the activation of caspase-3 and the cleavage of PARP protein. Therefore, cdk specific inhibitors provide a possible alternative therapeutic target for HIV-1 infection.

### DETAILED DESCRIPTION

Preferably, R₂ is a branched or unbranched alkyl radical substituted by one or more OH groups.

More preferably, R₂ is a branched or unbranched C₁₋₆ alkyl radical substituted by one or more OH groups.

More preferably, R₂ is NHCH(R₄)CH(R₃)OH wherein R₃ and R₄ are each independently H or alkyl.

Preferably, R₃ is H or methyl and R₄ is H, methyl, ethyl or propyl.

Even more preferably, R₂ is selected from NH—CH(CHMe₂)CH₂OH, NHCH₂CH₂OH and NHCH(CH₂CH₃)CH₂OH.

In one particularly preferred embodiment, R₂ is 2-hydroxymethylpyrrolidin-1-yl or 3-hydroxypiperidin-1-yl.

In a preferred embodiment, R₆ is an unsubstituted benzylamino or phenylamino group.

In an alternative preferred embodiment, R₆ is a benzylamino or phenylamino group substituted by one or more chloro or COOR′ groups.

In a particularly preferred embodiment, R₆ is an unsubstituted benzylamino group or a phenylamino group substituted by one or more chloro or COOR′ groups, where R′ is preferably H.

Even more preferably, R₆ is an unsubstituted benzylamino group or a 3-chlorophenylamino group or a 4-carboxy-3-chlorophenylamino group.

In another preferred embodiment, R₆ is selected from pyridyl-2-yl-methylamino, pyridyl-3-yl-methylamino and pyridyl-4-yl-methylamino.

Preferably, R₉ is a branched or unbranched C₁₋₆ alkyl group or a cyclopentanyl group.

Even more preferably, R₉ is methyl or isopropyl. In a particularly preferred embodiment of the invention, the compound of formula I is

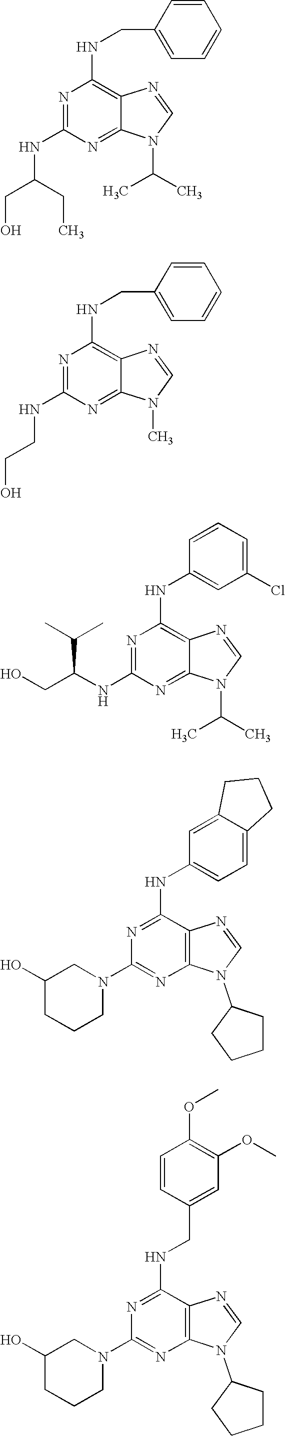

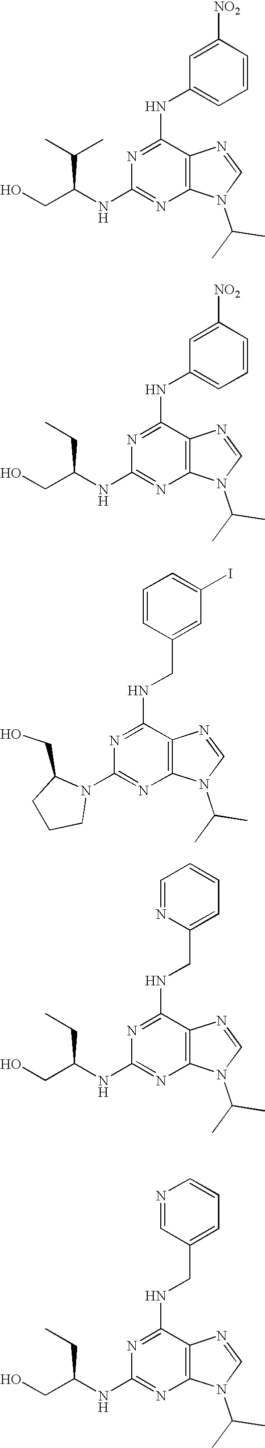

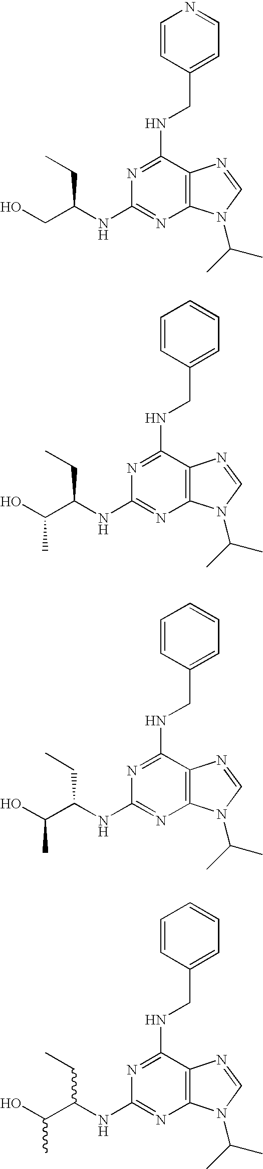

Even more preferably, the compound of formula I is

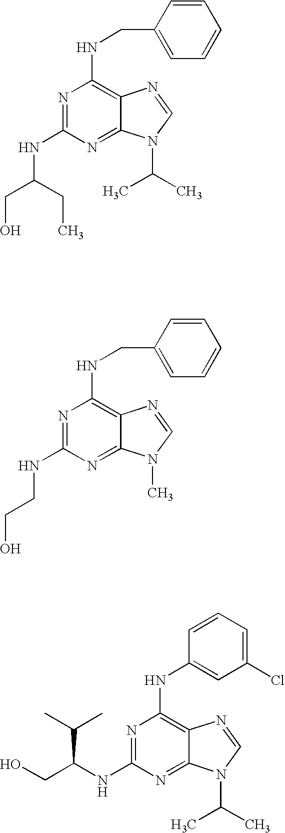

In one preferred embodiment, the autoimmune disorder is a viral infection.

In a particularly preferred embodiment, the autoimmune disorder is a HIV-1-related disorder.

Preferably, the compound of formula I inhibits HIV-1 replication

Even more preferably, the compound of formula I induces apoptosis in HIV-1 infected cells

In a preferred embodiment of the invention, the compound of formula I inhibits Cyclin A- and/or E-associated histone H1 kinase.

Even more preferably, the compound of formula I inhibits cdk7 and/or cdk9.

Preferably, the compound of formula I is used in the treatment of AIDS.

A second aspect of the invention relates to a method of treating an autoimmune disorder comprising administering to a subject in need thereof a therapeutically effective amount of a compound of formula I

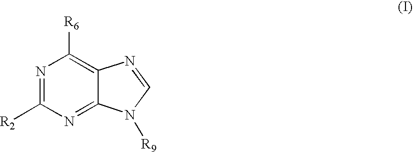

wherein

R₂ is R—NH wherein R is a branched or unbranched alkyl radical, a piperidinyl group or pyrrolidinyl group, each of which may be optionally substituted by one or more —OH, halogen, amino or hydroxyalkyl groups;

R₆ is phenylamino, benzylamino or pyridyl-methylamino, indan-5-amino, where in each case the aryl group may be unsubstituted or substituted by one or more —OR″, halogen, NO₂, amino or COOR′ groups, wherein R′ and R″ are each independently H or a branched or unbranched alkyl group; and

R₉ is a branched or unbranched alkyl group or a cycloalkyl group.

The preferred embodiments for the second aspect of the invention are identical to those described above in respect of the first aspect.

Pharmaceutical Salts

The compounds of the present invention may be administered as pharmaceutically acceptable salts. Typically, a pharmaceutically acceptable salt may be readily prepared by using a desired acid or base, as appropriate. The salt may precipitate from solution and be collected by filtration or may be recovered by evaporation of the solvent.

Pharmaceutically-acceptable salts are well known to those skilled in the art, and for example include those mentioned by Berge et al, in J.Pharm.Sci., 66, 1-19 (1977). Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

The compounds of the present invention may exist in polymorphic form.

In addition, the compounds of the present invention may contain one or more asymmetric carbon atoms and therefore exists in two or more stereoisomeric forms. The present invention includes the individual stereoisomers of the compound and, where appropriate, the individual tautomeric forms thereof, together with mixtures thereof.

An individual enantiomer of the compound may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The present invention also includes all suitable isotopic variations of the compounds or pharmaceutically acceptable salts thereof. An isotopic variation of a compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the compound and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the compound and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the present invention and pharmaceutically acceptable salts thereof of this invention can generally be prepared by conventional procedures using appropriate isotopic variations of suitable reagents.

The present invention also includes (wherever appropriate) the use of zwitterionic forms of the compounds of the present invention.

The terms used in the claims encompass one or more of the forms just mentioned.

Formulation

The component(s) of the present invention may be formulated into a pharmaceutical composition, such as by mixing with one or more of a suitable carrier, diluent or excipient, by using techniques that are known in the art.

Pharmaceutical Compositions

The present invention provides a pharmaceutical composition comprising a therapeutically effective amount of one or more compounds and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s). Examples of suitable carriers include lactose, starch, glucose, methyl cellulose, magnesium stearate, mannitol, sorbitol and the like. Examples of suitable diluents include ethanol, glycerol and water.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Where the composition is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Therapy

The agents identified by any such assay method may be used as therapeutic agents—i.e. in therapy applications.

As with the term “treatment”, the term “therapy” includes curative effects, alleviation effects, and prophylactic effects.

The therapy may be on humans or animals.

Administration

The components of the present invention may be administered alone but will generally be administered as a pharmaceutical composition—e.g. when the components are is in admixture with a suitable pharmaceutical excipient, diluent or carrier selected with regard to the intended route of administration and standard pharmaceutical practice.

For example, the composition can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical composition is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the compound may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracere-broventricular, intracerebral, subcutaneous, ophthalmic (including intravitreal or intracameral), transdermal, rectal, buccal, vaginal, epidural, sublingual.

Where the composition comprises more than one compound, it is to be understood that not all of the components of the pharmaceutical need be administered by the same route. Likewise, if the composition comprises more than one active component, then those components may be administered by different routes.

If a component of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component(s) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the component(s) of the present invention can be administered in the form of a suppository or pessary, or it may be applied topically in the form of a gel, hydrogel, lotion, solution, cream, ointment or dusting powder. The component(s) of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application, topically to the skin, the component(s) of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

In a preferred embodiment of the invention, the pharmaceutical composition is administered orally.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Depending upon the need, the agent may be administered at a dose of from 0.01 to 30 mg/kg body weight, such as from 0.1 to 10 mg/kg, more preferably from 0.1 to 1 mg/kg body weight.

Inhibition of HIV-1 Transcription in the Presence of cdk Inhibitors

We initially tested 14 different cdk inhibitors on both infected and uninfected T cells, among which three purine analogues, Olomoucine, Roscovitine, and Purvalanol A, were further selected primarily due to the lack of toxicity and the reversibility of inhibition on cdk's in uninfected T and monocytic cells (data not shown).

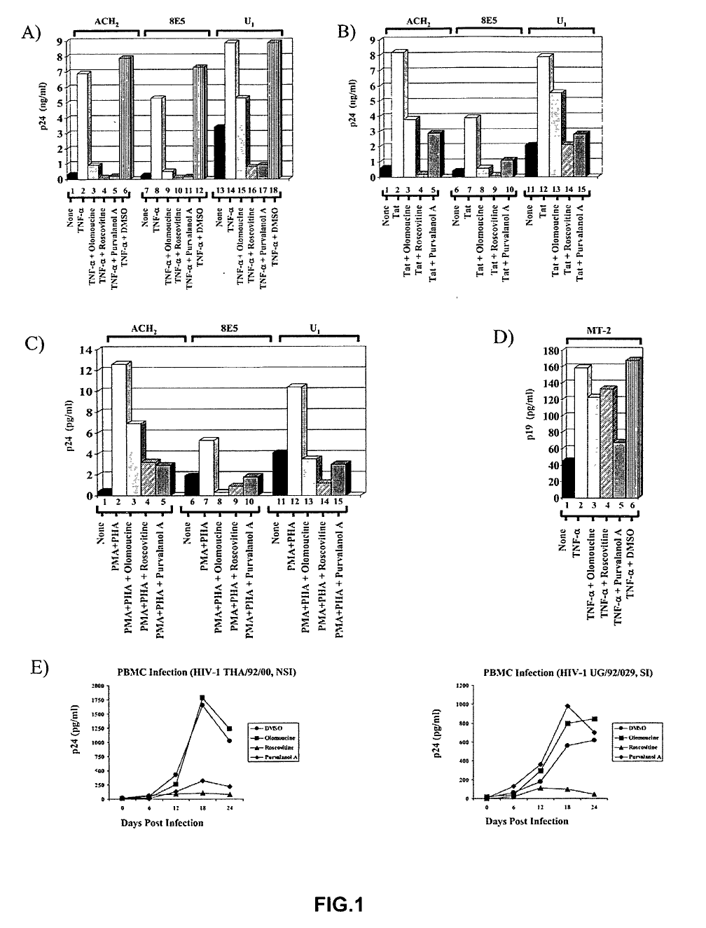

We chose HIV-1 stably integrated cells, lymphocytic ACH₂ and 8E5 cell lines, as well as monocytic U1 cells (and their uninfected parental counterparts CEM and U937, respectively) for our initial studies, since viral transcription could be activated and scored through signals, such as Tat, TNF-α or phorbol myristate acetate (PMA) and PHA. We first activated viruses in these cells with TNF-α (10 ng/ml) at 37° C. for 2 h, followed by washing, and then we incubated cells with 10 μM concentrations of each drug. The p24 concentration in the medium was determined by ELISA assay, and the results of such an experiment are shown in FIG. 1A. All drugs inhibited HIV-1 replication to various degrees, with Olomoucine (due to its highest IC₅₀ value) being the least effective and Roscovitine and Purvalanol A being the most effective of the three drugs. A somewhat similar pattern of drug inhibition was also seen when ACH₂, 8E5, and U1 cells were treated with either Tat or a combination of PMA and PHA to induce full-length viral transcripts. Tat-treated cells (FIG. 1B) were best inhibited in the presence of Roscovitine, whereas PHA- and PMA-treated cells were inhibited well with all three drugs (FIG. 1C). Interestingly, compared to other retrovirally infected cells such as MT-2 (infected with HTLV-1), only Purvalanol A inhibited HTLV-1 replication in these cells (FIG. 1D). We next examined the effect of all three drugs on primary HIV-1 field isolates of SI and NSI strains. Activated PBMC were infected with two independent HIV-1 viral strains of SI (UG/92/029, subtype A envelope) and NSI (THA/92/001, subtype E envelope). Cells were treated with 10 μM concentrations of each drug postinfection, and the p24 gag antigen level was determined by ELISA. The results of such an experiment are shown in FIG. 1E, in which Roscovitine (for both isolates) and Purvalanol A (only for NSI strain) effectively blocked viral replication.

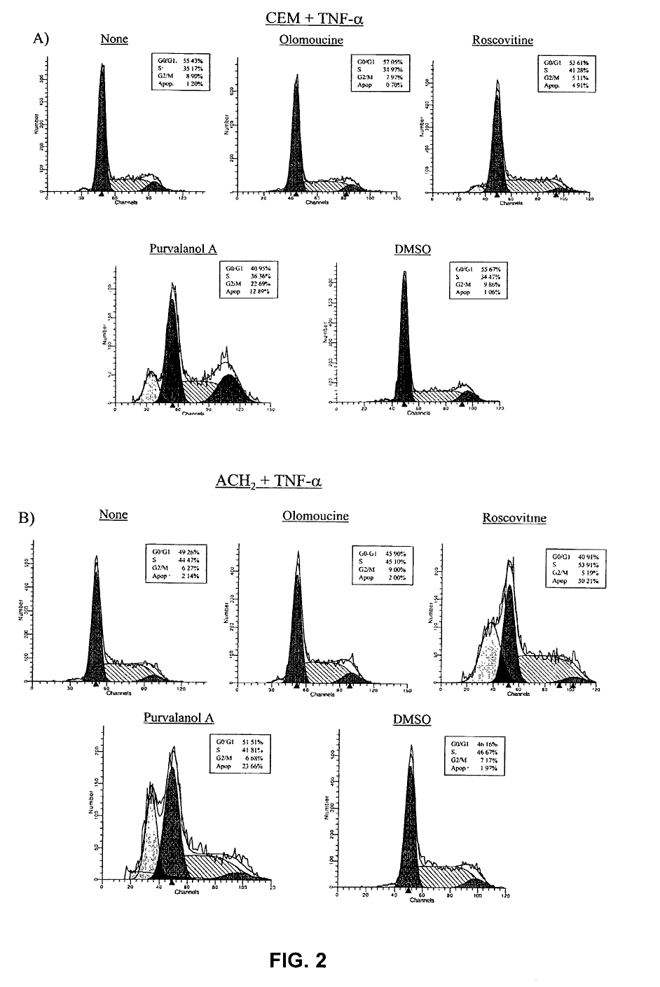

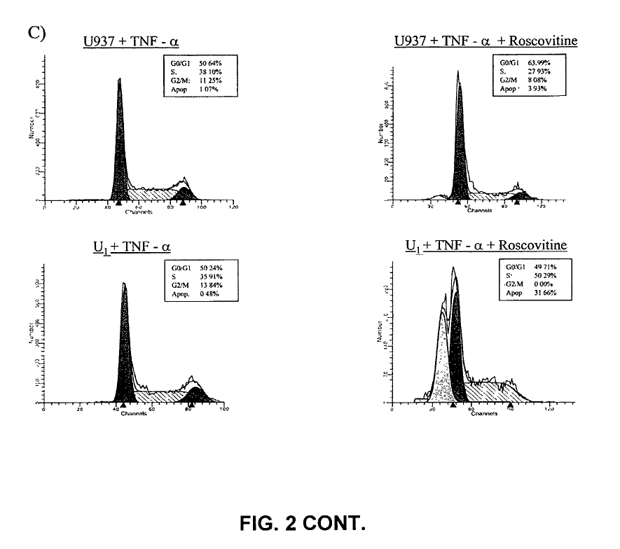

We next examined the cellular effects of these drugs on the cell cycle progression of HIV-1-infected and uninfected cells. Cells were treated with TNF-a washed, and grown in the presence of Olomoucine, Roscovitine, or Purvalanol A, and their cell cycle distributions were assessed 48 h after treatment. In uninfected CEM cells, at concentrations of 10 μM Olomoucine and Roscovitine did not change the cell cycle progression significantly, whereas Purvalanol A showed an increase in the G₂/M population and induced apoptosis (FIG. 2A). We next performed similar experiments on HIV-1-infected cells and observed increased apoptosis in the Purvalanol A- and Roscovitine-treated cells (FIG. 2B). Interestingly, Roscovitine selectively induced cell death in the HIV-1-infected cells (30.21% apoptosis in ACH₂ versus 4.9% in CEM cells). Therefore, the selective killing mechanism by this particular drug might partially be responsible for the decrease in HIV-1 titers observed in the infected cells. We also reasoned that Purvalanol A may be toxic to uninfected induced cells by increasing the G₂/M population and the increase of apoptosis, as is evident in FIG. 2A. Similar results were also observed in a set of promonocytic cell lines. U937 is the uninfected monocytic parental cell line, and U1 is the U937 cell line infected with two copies of integrated HIV-1, only one of which is wild type for viral progeny formation. Again, Roscovitine treatment of the uninfected parental cells showed no apparent apoptosis, whereas U1 cells showed an abundance of the apoptotic population (FIG. 2C). Collectively, these data imply that among all three drugs tested Roscovitine may be the best choice of an inhibitor for an induced HIV-1-infected cell. Roscovitine was able to selectively kill HIV-1 infected cells and inhibit viral production. This is in contrast to many apoptosis-inducing agents, such as TNF-α DNA-damaging agents (gamma irradiation, mitomycin C [6]), or sodium butyrate, in which cases the apoptosis of HIV-1-infected cell accompanies massive production and the release of infectious HIV-1 virions.

Inhibition of the Basal and Activated Transcription by cdk Inhibitors

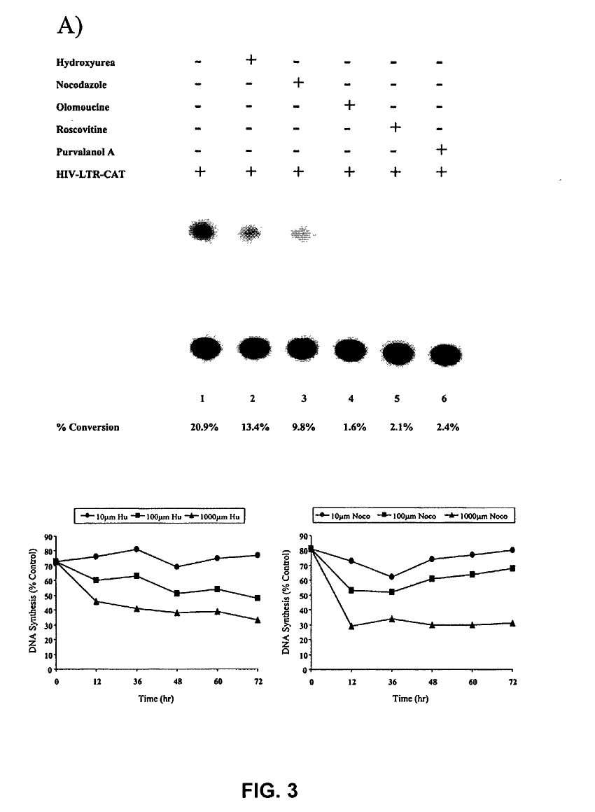

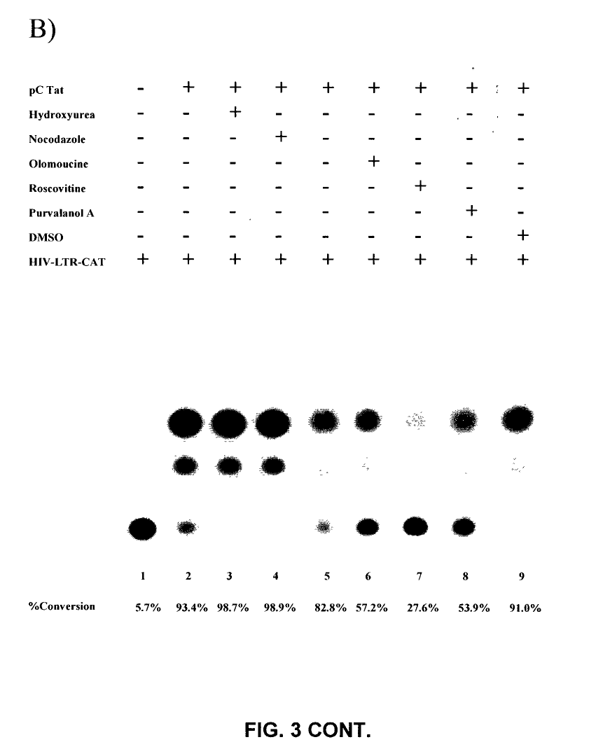

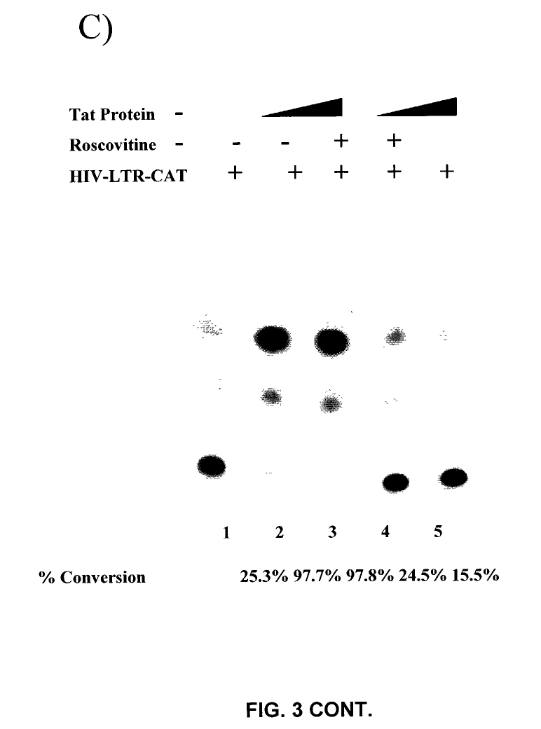

We next sought to determine whether the inhibition of HIV-1 transcription, observed above, could be mediated specifically by cdk inhibitors or by general cell cycle inhibitors. To distinguish between the two possibilities, we performed transfection experiments with all three cdk inhibitors tested above and two other well-established cell cycle inhibitors, namely, hydroxyurea and nocodazole. Hydroxyurea blocks DNA replication by inhibiting ribonucleotide reductase and thus arrests cell cycle progression at the G₁/S checkpoint, while nocodazole blocks at G₂/M by promoting tubulin depolymerization. When performing transfections in CEM cells, we observed a dramatic inhibition caused by Olomoucine, Roscovitine, and Purvalanol A and not by hydroxyurea or nocodazole at ca. 10 μM (FIG. 3A). As a control, the effects of both hydroxyurea and nocodazole were tested in CEM cells, where at ca. 10 μM no inhibition of DNA synthesis was observed (FIG. 3A, bottom). Therefore, the basal transcription of HIV-1 was inhibited by purine analogs and not general cell cycle inhibitors.

Activated transcription by Tat was also examined in CEM cells using the LTR-CAT reporter and pcTat (CMV-driven Tat). Consistent with basal transcription results, Tat transactivation was impaired by the cdk-specific drugs Olomoucine, Roscovitine, and Purvalanol A and not by hydroxyurea or nocodazole (FIG. 3B). Interestingly, Roscovitine inhibited activated transcription better than the other two analogs. To exclude the possibility that the reduction was mediated by inhibiting the CMV promoter driving the Tat gene, we performed a similar experiment with purified Tat protein and observed a similar inhibition of CAT activity by Roscovitine (FIG. 3C). Collectively, these results suggested that the cell cycle-dependent HIV-1 transcription requires cdk activities and, perhaps more importantly, general cell cycle inhibitors that do not target cdks do not inhibit HIV-1 postintegrative events.

Time Course Analysis of p24 Antigen Release in Roscovitine-Treated Cells

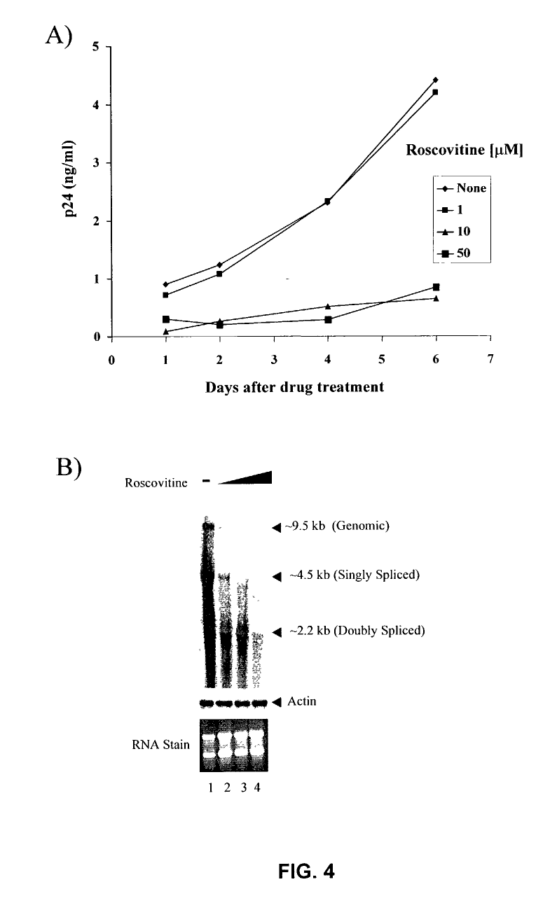

Based on the results obtained above, we decided to determine whether there was a time dependency in viral inhibition in ACH₂ cells treated with Roscovitine. The results of such an experiment is shown in FIG. 4A, where at a low concentration of 1 μM no obvious decrease of p24 was apparent; however, at 10 μM concentrations only low levels of viral p24 antigens were detected in the supernatant. Similar results were obtained in both U1 and 8E5 cells when treatment was continued for up to 21 days at either a 5 or a 10 μM concentration; in addition, neither hydroxyurea nor nocodazole was able to inhibit HIV-1 replication at these concentrations (data not shown).

To ensure that the inhibition was at the level of viral transcription and not at any other subsequent step, we performed Northern blot analysis using whole genomic HIV-1 probes. Viral transcripts of genomic (9.5-kb), structural (4.5-kb), and regulatory (2.2-kb) RNAs were all examined using a whole HIV-1 probe. The 9.5-kb genomic RNA is the precursor for the structural and regulatory mRNA and can be packaged into virions. As shown in FIG. 4B, when ACH₂ cells were treated with TNF-α and subsequently with increasing concentrations of Roscovitine, all genomic, structural, and regulatory RNAs were dramatically decreased, suggesting that the inhibition was indeed at the level of gene expression. A comprehensive count of all three classes of the RNAs showed downregulation of HIV-1 basal (doubly spliced regulatory RNAs) and activated (singly spliced structural RNAs, unspliced genomic RNA) transcription (data not shown).

Roscovitine Inhibits Cyclin E- and Cyclin A-Associated Histone H1 Kinase, as well as cdk7 and cdk9 Kinase Activities in HIV-1-Infected Cells

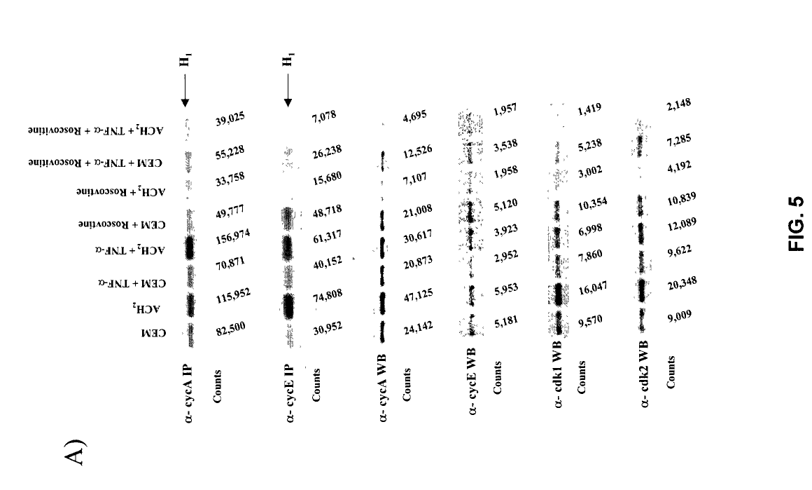

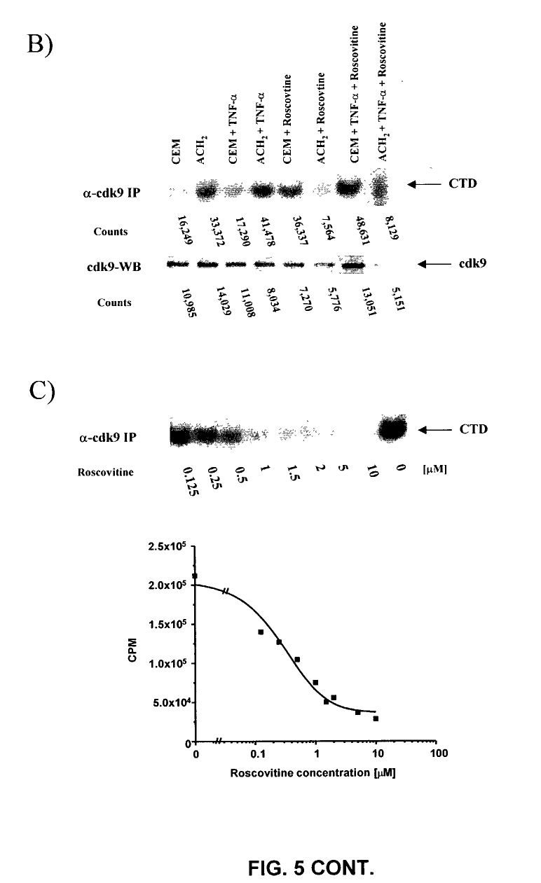

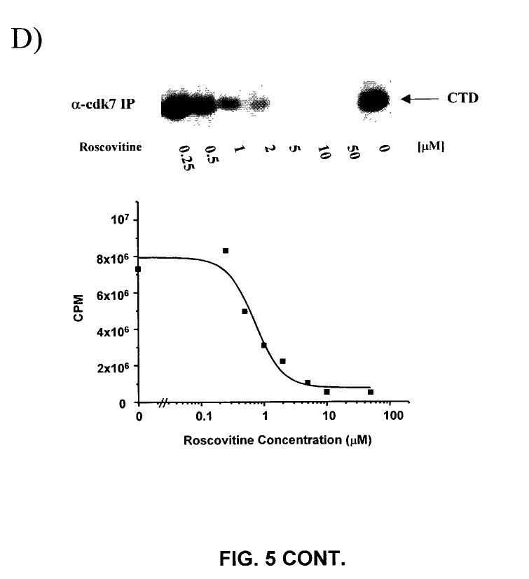

Roscovitine has been reported to inhibit cdk1, -2, and -5, but not cdk4 or -6, and the activity of this drug to date has not been reported on cdk3, -7, -8, or -9 (31). cdk9-cyclin T complex is a critical complex in the control of the Tat protein function (20, 51, 47), and cdk7 and -2 have also been shown to associate with the Tat complex (33). We therefore examined the effects of Roscovitine on cdk2, -7, and -9 activity using histone HI and RNA Pol II CTD peptides as in vitro substrates. Cyclin A or E immunoprecipitates were obtained from uninfected and infected cells that were treated with Roscovitine and used in kinase assays. The results of such an experiment are shown in FIG. 5A, where cyclin A or cyclin E immunoprecipitate showed an average of twofold induction from ACH₂ cells. Importantly, the addition of Roscovitine decreased the histone HI kinase activity from both cyclin immunoprecipitates in ACH₂ cells and not in control CEM cells. Similar reductions of cyclin A, cyclin E, cdk1, and cdk2 protein levels were also detected by Western blot analysis. Lower levels of these proteins in Roscovitine-treated cells might be due to the induced apoptosis or to the accelerated digestion of activated cyclin-cdk complexes in the infected cells. It is interesting to note that the transcription of cyclin A depends on the presence of an active cyclin E complex in the cell. The data in FIG. 5A indicate that the kinase activity of the cyclin E complex is lowered by more than eightfold (ACH₂+TNF-α versus ACH₂+TNF-α +Roscovitine) in HIV-1-infected and -induced cells. This may explain the observed lower cyclin A kinase activity and points to Roscovitine's primary effect on the cyclin E-associated complex. Alternatively, the levels of cdk 1 and 2 protein also show a ˜5-fold drop in the same extracts, which may indicate that Roscovitine selectively targets the cyclin-cdk complex in infected rather than in uninfected cells. The mechanism of this downregulation on cdk1 and 2 in infected cells remains to be determined; however, preliminary Northern blot data indicate that transcription of these cdk's is not affected by Roscovitine (data not shown).

We examined the effect of Roscovitine on cdk9-cyclin T complex from both infected and uninfected cells. cdk9-cyclin T immunoprecipitates were washed and mixed with an in vitro-synthesized CTD peptide for kinase assays. The result of such an experiment is shown in FIG. 5B, where the cdk9 activity in ACH₂ cells is ca. twofold higher than in CEM cells and, again, Roscovitine treatment led to a decrease of cdk9 levels and its activity on phosphorylation of the RNA Pol II CTD. We next performed an in vitro kinase assay on the cdk9 immunoprecipitates in the presence of various concentrations (0.1, 0.25, 0.5, 1, 1.5, 2, 5, or 10 μM) of Roscovitine. The substrate used in this experiment was the CTD peptide of RNA Pol II. As shown in FIG. 5C, the in vitro IC₅₀ value of Roscovitine on cdk9 was calculated to be ˜0.6 μM. This value was similar to the IC₅₀S that have been reported with cdk2-cyclin E (0.7 μM), cdk2-cyclin A (0.7 μM), and cdk1-cyclin B (0.65 μM) (31). Finally, we determined the IC₅₀ of Roscovitine inhibition on cdk7 kinase activity to phosophorylate the CTD peptide of RNA Pol II. As calculated from FIG. 5D, the in vitro IC₅₀ value was also ˜0.6 μM. Taken together, our data implies that all cdk's that might be involved in the transcriptional regulation of HIV-1 (cdk2, -7, and -9) were equally sensitive to Roscovitine treatment.

Roscovitine Selectively Sensitizes HIV-1-Infected Cells to Apoptosis

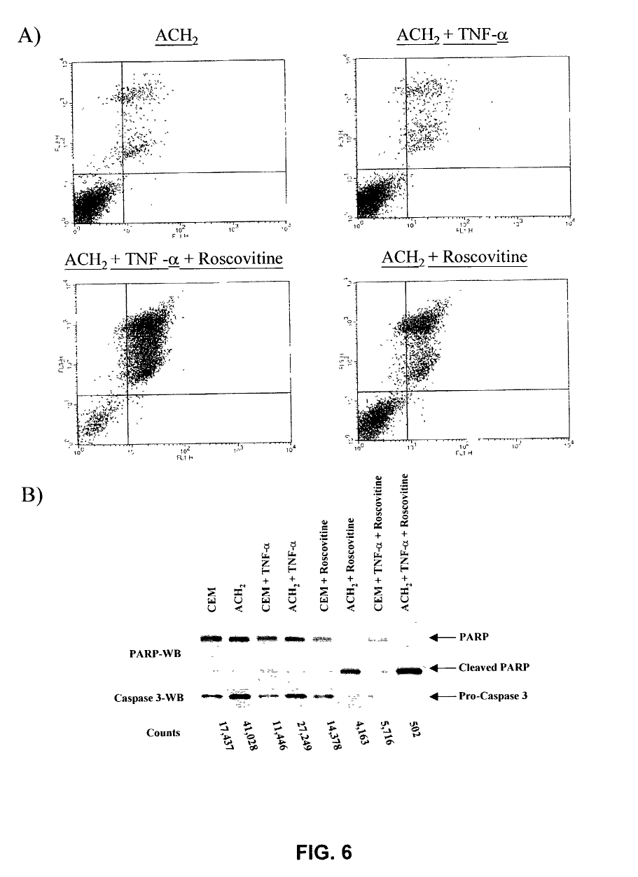

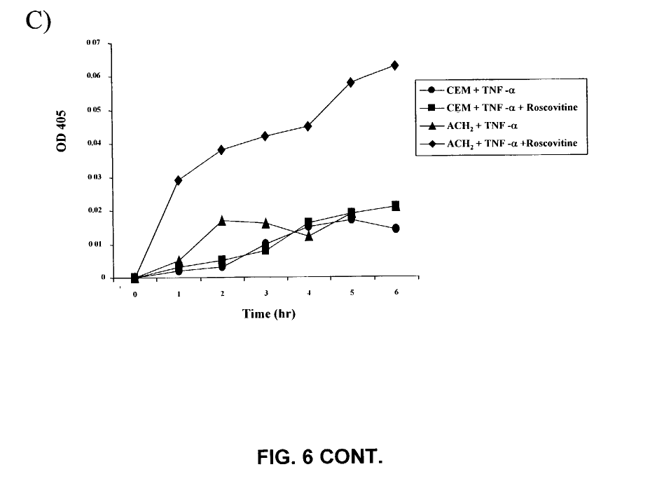

Apoptosis and necrosis are two pathways that lead to cell death. Apoptosis is characterized by a series of morphological features, including cell shrinkage, plasma membrane blebbing, phosphatidylserine translocation to the outer leaflet of the plasma membrane, nuclear condensation, and DNA fragmentation (1). To distinguish apoptotic from necrotic cells, we performed a flow cytometry experiment using Annexin V and PI double staining. Annexin V is a sensitive probe for phosphatidylserine, and PI was used to detect membrane loss, since membrane loss leads to the accessibility of PI staining to DNA. Exposure of HIV-1-infected ACH₂ cells to TNF-α alone did not result in an increase of Annexin V-stained cells; however, Roscovitine-treated cells, especially in presence of the TNF-α, resulted in a remarkable increase in the number of apoptotic cells. At 48 h after drug treatment, most of the apoptotic cells were found at the late stage of apoptosis and not in the necrotic population (FIG. 6A). We next investigated whether the apoptotic cascade through the caspase-3 pathway was activated under drug treatment. Caspase-3, a cysteine protease, is present in cells as an inactive procaspase-3 form and, in many cases, this enzyme is activated at the onset of apoptosis. We reasoned that cdk inhibitor treatment could result in an increase of the cleaved of procaspase-3 in HIV-1-infected cells, thus increasing the caspase-3 activity on substrates such as PARP. PARP is a 1112-kDa nuclear protein, which specifically has been shown to be cleaved by caspase-3. PARP is a protein necessary for the ribosylation of a number of critical substrates in DNA damage checkpoint, including p53, DNA-PK, PCNA, DNA polymerase alpha and beta, topoisomerase I and II, and RNA Pol I and II, as well as histones and lamins (2). In immunoblotting experiments, we observed that PARP was almost completely cleaved in induced ACH₂ cells when exposed to Roscovitine, implying that caspase-3 is active in drug-treated cells (FIG. 6B). Finally, to further control for the caspase-3 activity, we utilized treated lysates with the caspase-3 substrate, DEVD-pNA. Briefly, cells were lysed, and equal amounts of lysates were incubated with the caspase-3 substrate, DEVD-pNA, at 37° C. for 3 h. Absorbances of the samples were read every 60 min in a Spectramax 250 microplate reader at 405 nm. The result of such an experiment is shown in FIG. 6C, in which induced ACH₂ cells showed an average of ˜5-fold-higher activity when treated with Roscovitine.

Discussion

In this study, we have demonstrated that HIV-1 transcription requires cellular cdk's, namely, cdk2, -7, and -9. cdk's may function in at least two ways: to control HIV-1 transcription and to keep HIV-1-infected cells alive. Exposure of HIV-1-infected cells to cdk-specific inhibitors, such as Roscovitine, resulted in the loss of HIV-1 transcription and the induction of apoptosis in HIV-1-infected cells. More importantly, the apoptosis was not seen in uninfected control cells. These two mechanisms may be responsible for the inhibitory effects exerted-by Roscovitine in HIV-1 progeny formation. When examining the effects of Roscovitine on primary HIV-1 SI and NSI field isolates, we found that activated PBMC infected with either of these two viruses did not support viral replication.

A number of studies have demonstrated that hydroxyurea can inhibit HIV-1 replication by reducing the intracellular pool of deoxynucleotides, which is essential for successful reverse transcription of HIV-1 RNA in both activated and resting PBMC (12, 28, 29, 30). However, in this study we examined the postintegrative events related to HIV-1 transcription and the subsequent steps prior to progeny formation. The postintegrative HIV-1 progeny formation remained essentially the same in the presence of hydroxyurea and was slightly higher in the G₂/M cells when blocked with nocodazole (data not shown). Since the treatment of cells with hydroxyurea or nocodazole (at low concentrations) did not decrease the HIV-1 replication and since the requirement of efficient HIV-1 transcription in G₁ or G₂ phase is limited to the availability of cdk's, cell cycle blockers that do not specifically target cdk's would not inhibit HIV-1 postintegrative events.

It is important to note that our current study does not address issues related to the initiator and effector caspases involved in the apoptosis of infected cells in sufficient detail. Also, events related to the apoptosis unfold too rapidly to determine their temporal sequence in HIV-1-infected cells. However, some of our preliminary studies suggest that caspase-3, -7, and -8 are activated in infected cells and may be responsible for the apparent apoptosis in both treated ACH₂ and U1 cells. The activation is further evident from the release of cytochrome c, from Western blots of the activated caspase-3, -7, and -8, from the presence of the Smac-DIABLO complex with the mXIAP (inhibitor of caspase activity) as detected by immunoprecipitations followed by Western blotting, and from the inhibition of caspase activity seen with z-VAD-fmk peptide. Furthermore, substrates that have tested positive for caspase-3 (the executioner) activity in both ACH₂ and U 1 cells were Rb, SREBP-1, heteroribonuclear protein Cl, PKC, DNA-PK_{cs}, U1-70, and PARP (L. Deng and F. Kashanchi, unpublished results). Therefore, the apparent apoptosis of the infected cells following the addition of the cell cycle inhibitor, Roscovitine, may ultimately be linked to mitochondrial dysfunction, but the exact sequence of events leading to apoptosis awaits further detailed analysis and experimentation.

Currently, clinical treatment of AIDS patients with a combination of anti-HIV-1 drugs has been successful in reducing the viral load in the bloodstream. However, eradication of the long-lived chronically and latently infected cells cannot be achieved by highly active antiretroviral therapy (HAART) (33). In addition, reverse transcriptase and protease inhibitors do not block virus particle production in latently infected cells; rather, they act by preventing de novo infection. In this study, our data suggest that purine-derived cdk inhibitors have the potential for novel anti-HIV-1 therapy. Our assumption is based on the following rationales. (i) The transcription of newly synthesized HIV-1 RNAs in activated cells could be inhibited by cdk inhibitors and, for the first time, we show that cdk9-cyclin T and cdk7-cyclin H, which are required for HIV-1 transcription, can effectively be inhibited by Roscovitine. Functionally, Roscovitine inhibits HIV-1 transcription because the LTR requires and utilizes more than one cdk for its robust activated transcription, a scenario that may be unique to viral and not so much to cellular promoters. (ii) Roscovitine was able to induce apoptosis selectively in the HIV-1-infected cells and not in uninfected cells. (iii) Targeting cellular proteins and selective killing of HIV-1-infected host cells may be an effective method to prevent development of resistant viruses, which is a novel approach to eradicate latently and chronically infected cells. Finally, we recently have tested similar cdk inhibitors on other human and primate retroviruses (including Simian immunodeficiency virus, HIV-2, and HTLV-1), as well as on HHV-8, and found that these viruses were sensitive only to a select set of cdk inhibitors (M. Healey, D. Wang, and F. Kashanchi, unpublished results). Therefore, we predict that most viruses that have cell cycle stimulatory functions and require active cdk's for their survival may be targets for these drugs. Future experiments will determine whether cdk inhibitors are effective blockers in Simian/human immunodeficiency virus animal models.

The present invention is further described by way of example and with reference to the following figures wherein:

FIG. 1 shows the inhibition of HIV-1 by various cdk inhibitors. (A) HIV-1 latently infected cells (ACH₂, 8E5, and U1) were grown to the mid-log phase, treated with TNF-α (10 ng/ml) for 2 h, washed, and subsequently incubated with a 10 μM concentration of each cdk inhibitor. Five days later, the supernatants were collected, and the amount of newly synthesized HIV-1 was measured by p24 antigen assay. (B) Purified Tat (300 μg) was electroporated into ACH₂, 8E5, and U1 cells and subsequently treated with various drugs for 5 days. (C) Experiment similar to that in panel A, except that both T and monocytic cell lines were treated with a combination of PMA (1 ng/ml) and PHA (1 μg/ml) for 12 h for induction of virus. (D) Experiment similar to that in panel A, except that MT-2 cells (HTLV-1 infected) were treated with TNF-α (10 ng/ml) for 2 h, washed, and subsequently treated with one of the three cdk inhibitors. Seven days later samples were collected and used for p19 gag ELISA. (E) PHA-activated PBMC (5×10⁶) were infected with HIV-1 either SI (UG/92/029 Uganda strain, subtype A envelope) or NSI (THA/92/001, Thailand strain, subtype E envelope) strains. Unadsorbed viruses were washed after 6 h, and cells were cultured in fresh media with various cdk inhibitors (10 μM). Samples were collected every sixth day and used for p24 gag ELISA assay.

FIG. 2 shows cell cycle analysis of infected and uninfected cells following cdk inhibitor treatment. (A) CEM (uninfected) cells were treated with TNF-α (10 ng/ml) for 2 h and then grown in the presence of a 10 μM concentration of each cdk inhibitor. Forty-eight hours later, cells were collected, fixed with 70% ethanol, PI stained, and analyzed by FACS. (B and C) Experiments similar to the experiment in panel A were performed in ACH₂ (B) or U1 (C) cells (both latently HIV-1 infected) plus TNF-α, followed by ethanol fixation, PI staining, and analysis by FACS. Apoptosis (Apop) represents a mixture of cells from either G₁, S, or G₂/M with characteristics of cell death.

FIG. 3 discloses inhibition of HIV-1 basal and activated transcription by cdk inhibitors. (A) CEM (12D7) cells were transfected with CAT reporter gene driven by HIV-1 LTR and then incubated for 20 h with a 10 μM concentration of either hydroxyurea, nocodazole, Olomoucine, Roscovitine, or Purvalanol A. CAT assays were performed using 2 μg of cellular extract from each sample. The graphs at the bottom of the figure represent the effect of various concentrations of general cell cycle inhibitors (hydroxyurea and nocodazole) on CEM cells. (B) Inhibition of activated transcription in CEM cells was examined using transfected HIV-1 LTR-CAT, together with pcTat plasmid. (C) Experiment similar experiment to that in panel B, except that pcTat plasmid was replaced with E. coli-expressed and purified Tat protein

FIG. 4 shows a time course study of HIV-l progeny formation in the presence of Roscovitine. (A) ACH₂ (HIV-1 latently infected) cells were induced and grown in the presence of 1, 10, or 50 μM Roscovitine. At days 2, 4, and 6, the supernatants were collected, and the released HIV-1 particles were measured by using a p24 antigen ELISA assay. (B) Twenty micrograms of total RNA was separated on an RNA-formaldehyde-agarose gel, transferred, and probed with full-length labeled HIV-1 genome RNA. The hybridized genomic RNA, singly spliced (structural), and doubly spliced (regulatory) RNAs were exposed and counted by using Molecular Dynamic PhosphorImager software. All samples were initially treated with TNF-α, and lanes 1 to 4 contained either DMSO or 1, 10, or 50 μM Roscovitine, respectively. Both actin probe hybridization and ethidium bromide staining of the gel are shown below.

FIG. 5 shows the inhibition of kinase activities by Roscovitine in HIV-1-infected and uninfected cells. ACH₂ (HIV-1-infected) or CEM (uninfected) cells treated with TNF-α (10 ng/ml) for 2 h were washed and incubated with 10 μM concentrations of each of the cdk inhibitors. Twenty-four hours later, the cells were harvested and lysed in lysis buffer (see Materials and Methods). The protein concentration was quantified by use of the BioRad protein concentration kit and also by Coomassie blue staining after on 4 to 20% Tris-glycine PAGE. (A) A total of 1 mg of cellular extract was used for immunoprecipitation with 5 μg of cyclin A or E antibody and washed twice with TNE 150 plus 0.1% NP-40 and twice with the kinase buffer. The cyclin A- and E-associated cdk2 kinase activity was determined, using histone H1 as a substrate. The cdk1, cdk2, cyclin A, and cyclin E levels were analyzed on a PVDF membrane probed with specific antibodies and detected with ¹²⁵I-labeled protein G. The counts represent quantitation of the autoradiographic bands using the PhosphorImager software program. (B) Immunoprecipitation experiments similar to those shown in panel A were performed, followed by assay for cdk9 kinase activity using the CTD dipeptide from RNA Pol II. (C) In vitro titration of cdk9 inhibition by Roscovitine. Cdk9 protein complex was immunoprecipitated from CEM cells, and the enzymatic activity was assayed in the presence of increasing concentrations of Roscovitine. (D) Active cdk7 protein complex was immunoprecipitated from CEM cells, and the IC₅₀ value was determined as in panel C.

FIG. 6 shows that Roscovitine treatment induces apoptosis in HIV-1-infected cells. (A) ACH₂ (HIV-1 latently infected) cells were treated with TNF-α (10 ng/ml) for 2 h and then grown in the presence of 10 μM Roscovitine for 48 h. Cells were collected, stained with PI and FITC-Annexin V, and analyzed by FACS. The lower left, upper left, lower right, and upper right panels represent the normal, necrotic, early-apoptotic, and late-apoptotic cells, respectively. (B) Cellular extracts from Roscovitine-treated and untreated cells were resolved on a 4 to 20% Tris-glycine gel and transferred to a PVDF membrane. Membranes were then probed with rabbit polyclonal anti-caspase-3 or anti-PARP antibody (Santa Cruz). The antigen-antibody complexes were detected using ¹²⁵I-labeled protein G. (C) Cells were washed in PBS and analyzed for caspase-3 activity using a colorimetric protease assay kit (Chemicon). Lysates were incubated with the caspase-3 substrate, 200 μM DEVD-pNA, at 37° C. for 3 h. Absorbances of samples were read every 60 min in a microplate reader at 405 nm. Symbols: ● CEM+TNF-α; ▪ CEM+TNF-α+Roscovitine; ▴ ACH₂+TNF-α; ♦ ACH₂+TNF-α+Roscovitine.

### EXAMPLES

Cell Culture, Peptides, Plasmids, Antibodies, and Drugs

ACH₂ (7, 9) and 8E5 (10) cells are both HIV-1-infected lymphocytic cells, with the integrated wild-type single-copy (ACH₂) reverse transcriptase and an integrated single-copy reverse transcriptase-defective virus (8E5) in CEM (12D7) cells (36). The CEM T cell (12D7) is the parental cell for both ACH₂ and 8E5 cells. U1 is a monocytic clone harboring two copies of the viral genome (10) from parental U973 cells. MT-2 cells are infected with several copies of human T-cell leukemia virus type 1 (HTLV-1) and produce full-length viral particles. All cells were cultured at 37° C. with up to 10⁵ cells per ml in RPMI 1640 media containing 10% fetal bovine serum and treated with a mixture of 1% streptomycin and penicillin antibiotics and 1% L-glutamine (Gibco-BRL).

Plasmids of HIV-LTR-CAT and pcTat were described previously (6). The Tat protein was produced in Escherichia coli and purified using Sephacryl S-200, followed by C₁₈ reversed-phase high-pressure liquid chromatography (24). The purified Tat protein was then dried and resuspended in phosphate-buffer saline (PBS) containing 1 mM dithiothreitol (DTT) and 0.01% bovine serum albumin. Wild-type C-terminal domain (CTD) peptide was a generous gift from M. Morange (45). Antibodies against cdk1 (C-19), cdk2 (M-2), cdk7 (C-19), cdk9 (L-19), cyclin E (M-20), cyclin A (H-432), poly(ADP-ribose) polymerase PARP (N-20), and caspase-3 (H-277) were purchased from Santa Cruz Biotechnology. The cdk inhibitors Olomoucine, Roscovitine, and Purvalanol A were synthesized in house and also purchased from Calbiochem and dissolved in dimethyl sulfoxide (DMSO) in 10 mM stock concentrations.

Lymphocyte Transfection and CAT Assays

Lymphocyte CEM (12D7) cells were grown to mid-log phase and were processed for protein electroporation according to a previously published procedure (24). Only one modification was introduced, in which the cells were electroporated at 230 V and plated in 10 ml of complete RPMI 1640 medium for 18 h prior to harvest and chloramphenicol acetyl transferase (CAT) assay. For CAT assays, transfected cells were harvested, washed once with PBS without Ca²⁺ and Mg²⁺, pelleted, and resuspended in 150 μl of 0.25 M Tris (pH 7.5). Cells were freeze-thawed three times with intermittent vortexing and then incubated for 3 min at 68° C., followed by centrifugation. Supernatants were transferred to 1.5-ml Eppendorf tubes and centrifuged, and the supernatants were used for the determination of protein concentration. CAT assays were performed with 2 μg of protein according to the method of Gorman et al. (15).

Cell Extract Preparation and Kinase Assays

Cells that were cultured to mid-log phase of growth were treated with or without tumor necrosis factor alpha (TNF-α) (10 ng/ml) for 2 h, washed with PBS without Ca²⁺ and Mg²⁺, and incubated for 48 h prior to lysis in a buffer containing 50 mM Tris-HCl (pH 7.5), 120 mM NaCl, 5 mM EDTA, 50 mM NaF, 0.2 mM Na₃VO₄, 1 mM DTT, 0.5% NP-40, and protease inhibitors (Protease Inhibitor Cocktail Tablets; Boehringer Mannheim [one tablet per 50 ml]). Kinases from immunoprecipitated associated complexes were then assayed by the transfer of phosphate from [γ-³²P]ATP to the substrates histone HI or peptide representing the CTD of RNA polymerase II (Pol 11) (45) in a reaction buffer consisting of 50 mM Tris (pH 7.4), 10 mM MgCl₂, 1 mM DTT, and 144 μM ATP (40 μCi of [γ-³²P]ATP). Reactions were performed at 37° C. for 30 min and stopped by the addition of sodium dodecyl sulfate (SDS) sample buffer. Samples were boiled for 5 min at 95° C., and the histone HI proteins were separated on a 4 to 20% Tris-glycine gel; the CTD peptides were separated on a 20% discontinuous SDS-polyacrylamide gel electrophoresis (PAGE) gel. Gels were autoradiographed, and the bands were counted using Molecular Dynamics PhosphorImager software.

Immunoblotting

Cells were pelleted by centrifugation, washed with PBS without Ca²⁺ and Mg²⁺, and lysed with lysis buffer as described above. The lysate was incubated on ice for 15 min and microcentrifuged at 4° C. for 10 min. Total cellular protein was separated on 4 to 20% Tris-glycine gels (Novex, Inc.) and transferred to polvinylidene difluoride (PVDF) membranes (Immobilon-P Transfer Membranes; Millipore Corp.) overnight at 0.08 A. Following the transfer, the blots were blocked with 5% nonfat dry milk in 50 ml of TNE 50 (100 mM Tris-Cl [pH 8.0], 50 mM NaCl, 1 mM EDTA) plus 0.1% NP-40. Membranes were probed with a 1:200 to 1:1,000 dilution of antibodies at 4° C. overnight, followed by three washes with TNE 50 plus 0.1% NP-40. The next day, the blots were incubated with 10 ml of ¹²⁵I-labeled protein G (Amersham; 50-μl/10 ml solution) in TNE 50 plus 0.1% NP-40 for 2 h at 4° C. Finally, the blots were washed twice in TNE 50 plus 0.1% NP-40 and placed on a PhosphorImager cassette for further analysis.

Flow Cytometry

For cell cycle analysis, cells treated with or without drugs were collected by low-speed centrifugation and washed with PBS without Ca²⁺ and Mg²⁺ and then fixed with 70% ethanol. For fluorescence-activated cell sorting (FACS) analysis, cells were stained with a cocktail of propidium iodide (PI) buffer (PBS with Ca²⁺ and Mg²⁺, RNase A [10 μg/ml], NP-40 [0.1%], and PI [50 μg/ml]) followed by cell-sorting analysis. FACS data acquired were analyzed by ModFit LT software (Verity Software House, Inc.).

Apoptosis was determined by using Annexin V and PI double staining (Annexin V-FITC; PharMingen International). Cells were washed twice with cold PBS without Ca²⁺ and Mg²⁺; resuspended in 1× binding buffer (10 mM HEPES-NaOH, pH 7.4; 140 mM NaCl; 2.5 mM CaCl₂), 2.5 μl of Annexin V-FITC, and 5 μl of PI/10⁵ cells; and incubated at room temperature for 15 min. Cells were acquired and analyzed using CELLQuest software (Becton Dickinson).

Caspase-3 Assay

Cells were washed in PBS and analyzed for caspase-3 activity by using a CPP32/Caspase-3 Colorimetric Protease Assay Kit (Chemicon, Temecula, Calif.) according to the manufacturers' instructions. Briefly, cells were lysed in 150 μl of cell lysis buffer provided in the kit. Protein concentrations of the lysates were determined by using the bicinchoninic acid assay reagent (Pierce, Rockford, Ill.). Equal amounts of lysates were incubated with the caspase-3 substrate, 200 μM DEVD-pNA, at 37° C. for 3 h. Absorbances of the samples were read every 60 min in a Spectramax 250 (Molecular Dynamics) microplate reader at 405 nm. Caspase-3 activity was proportional to the optical density at 405 nm.

Cell Proliferation

Cells (CEM) were initially treated with various concentrations of hydroxyurea or nocodazole and evaluated after 0, 12, 24, 48, 60, and 72 h. Subsequently, cells were incubated with 10 μCi of [³H]thymidine (Amersham) for 2 h prior to the end of each interval and harvested in an automatic cell harvester. The amount of radioactivity incorporated into the DNA was measured in a liquid scintillation counter (Packard) and expressed as the counts per minute (cpm). Data represented at the bottom of FIG. 3A are an average of three independent experiments.

Northern Blots

Total cellular RNA was extracted using the RNAzol reagent (Gibco-BRL). Total RNA (20 μg) was isolated 12 or 24 h posttreatment and run on a 1% formaldehyde-agarose gel overnight at 75 V, transferred onto a 0.2-μm (pore-size) nitrocellulose membrane (Millipore, Inc.), UV cross-linked, and hybridized overnight at 42° C. with ³²P-end-labeled HIV-1 full genomic RNA (Loftstrand, Gaithersburg, Md.). The next day, membranes were washed two times for 15 min each with 10 ml of 0.2% SDS-2×SSC (1×SSC is 0.15 M NaCl plus 0.015 M sodium citrate) at 37° C., exposed, and counted on a PhosphorImager cassette.

PBMC Infection

Phytohemagglutinin-activated PBMC were kept in culture for 2 days prior to each infection. Isolation and treatment of PBMC were performed by following the guidelines of the Centers for Disease Control (5a). Approximately 5×10⁶ PBMC were infected with either an SI (UG/92/029 Uganda strain, subtype A envelope, 5 ng of p24 gag antigen) or an NSI (THA/92/001, Thailand strain, subtype E envelope, 5 ng of p24 gag antigen) strain of HIV-1. Both viral isolates were obtained from the National Institutes of Health (NIH) AIDS Research and Reference Reagent Program (catalog numbers 1650 for strain UG192/029 and 1651 for strain THA/92/001). After 8 h of infection, cells were washed, and fresh media were added. Drug treatment was performed (only once) immediately after the addition of fresh media. Samples were collected every sixth day and stored at 20° C. for p24 gag enzyme-linked immunosorbent assay (ELISA).

HIV-1 p24 and HTLV-1 p19 ELISA

Media from HIV-1 infected cell lines were centrifuged to pellet the cells, and supernatants were collected and diluted to 1:100 to 1:1,000 in RPMI 1640 prior to ELISA. Supernatants from the infected PBMC were collected and used directly for the p24 antigen assay. The p24 gag antigen level was analyzed by using the HIVAG-1 Monoclonal Antibody Kit (Abbott Laboratories, Diagnostics Division). The HTLV-1 p19 core antigen ELISA kit was from Retro-Tek (Cellular Products).

Various modifications and variations of the described methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

### REFERENCES

1. Allen, R. T., W. J. Hunter III, and D. K. Agrawal. 1997. Morphological and biochemical characterization and analysis of apoptosis. J. Pharmacol. Toxicol. Methods 37:215-228.

2. Alvarez-Gonzalez, R., T. A. Watkins, P. K. Gill, J. L. Reed, and H. Mendoza-Alvarez. 1999. Regulatory mechanisms of poly(ADP-ribose) polymerase. Mol. Cell. Biochem. 193:19-22.

3. Barre-Sinoussi, F., J. C. Chermann, F. Rey, M. T. Nugeyre, S. Chamaret, J. Gruest, C. Dauguet, C. Axler-Blin, F. Vezinet-Brun, C. Rouzioux, W. Rozenbaum, and L. Montagnier. 1983. Isolation of a T-lymphotropic retrovirus from a patient at risk for acquired immune deficiency syndrome (AIDS). Science 220:868-871

4. Bresnahan, W. A., I. Boldogh, P. Chi, E. A. Thompson, and T. Albrecht. 1997. Inhibition of cellular Cdk2 activity blocks human cytomegalovirus replication. Virology 231:239-247

5. Bukrinsky, M. I., N. Sharova, M. P. Dempsey, T. L. Stanwick, A. G. Bukrinskaya, S. Haggerty, and M. Stevenson. 1992. Active nuclear import of human immunodeficiency virus type 1 preintegration complexes. Proc. Natl. Acad. Sci. USA 89:6580-6584. 5a. Centers for Disease Control. 1991. Isolation, culture, and identification of HIV: procedural guide. Centers for Disease Control, Atlanta, Ga.

6. Clark, E., F. Santiago, L. Deng, S. Chong, C. de La Fuente, L. Wang, P. Fu, D. Stein, T. Denny, V. Lanka, F. Mozafari, T. Okamoto, and F. Kashanchi. 2000. Loss of G₁/S checkpoint in human immunodeficiency virus type 1-infected cells is associated with a lack of cyclin-dependent kinase inhibitor p21/Wafl. J. Virol. 74:5040-5052

7. Clouse, K. A., D. Powell, I. Washington, G. Poli, K. Strebel, W. Farrar, P. Barstad, J. Kovacs, A. S. Fauci, and T. M. Folks. 1989. Monokine regulation of human immunodeficiency virus-1 expression in a chronically infected human T cell clone. J. Immunol. 142:431-438

8. Cujec, T. P., H. Okamoto, K. Fujinaga, J. Meyer, H. Chamberlin, D. O. Morgan, and B. M. Peterlin. 1997. The HIV transactivator TAT binds to the CDK-activating kinase and activates the phosphorylation of the carboxy-terminal domain of RNA polymerase II. Genes Dev. 11:2645-2657

9. Folks, T. M., K. A. Clouse, J. Justement, A. Rabson, E. Duh, J. H. Kehrl, and A. S. Fauci. 1989. Tumor necrosis factor alpha induces expression of human immunodeficiency virus in a chronically infected T-cell clone. Proc. Natl. Acad. Sci. USA 86:2365-2368

10. Folks, T. M., J. Justement, A. Kinter, C. A. Dinarello, and A. S. Fauci. 1987. Cytokine-induced expression of HIV-1 in a chronically infected promonocyte cell line. Science 238:800-802

11. Gallo, R. C., S. Z. Salahuddin, M. Popovic, G. M. Shearer, M. Kaplan, B. F. Haynes, T. J. Palker, R. Redfield, J. Oleske, B. Safai, et al. 1984. Frequent detection and isolation of cytopathic retroviruses (HTLV-III) from patients with AIDS and at risk for AIDS. Science 224:500-503

12. Gao, W. Y., A. Cara, R. C. Gallo, and F. Lori. 1993. Low levels of deoxynucleotides in peripheral blood lymphocytes: a strategy to inhibit human immunodeficiency virus type 1 replication. Proc. Natl. Acad. Sci. USA 90:8925-8928

13. Garriga, J., J. Peng, M. Parreno, D. H. Price, E. E. Henderson, and X. Grana. 1998. Upregulation of cyclin T1/CDK9 complexes during T cell activation. Oncogene 17:3093-3102

14. Goh, W. C., M. E. Rogel, C. M. Kinsey, S. F. Michael, P. N. Fultz, M. A. Nowak, B. H. Hahn, and M. Emerman. 1998. HIV-1 Vpr increases viral expression by manipulation of the cell cycle: a mechanism for selection of Vpr in vivo. Nat. Med. 4:65-71

15. Gorman, C. M., L. F. Moffat, and B. H. Howard. 1982. Recombinant genomes which express chloramphenicol acetyltransferase in mammalian cells. Mol. Cell. Biol. 2:1044-1051

16. Gray, N. S., L. Wodicka, A. M. Thunnissen, T. C. Norman, S. Kwon, F. H. Espinoza, D. O. Morgan, G. Barnes, S. LeClerc, L. Meijer, S. H. Kim, D. J. Lockhart, and P. G. Schultz. 1998. Exploiting chemical libraries, structure, and genomics in the search for kinase inhibitors. Science 281:533-538

17. Gray, N., L. Detivaud, C. Doerig, and L. Meijer. 1999. ATP-site directed inhibitors of cyclin-dependent kinases. Curr. Med. Chem. 6:859-875

18. Gummuluru, S., and M. Emerman. 1999. Cell cycle- and Vpr-mediated regulation of human immunodeficiency virus type 1 expression in primary and transformed T-cell lines. J. Virol. 73:5422-5430

19. He, J., S. Choe, R. Walker, P. Di Marzio, D. O. Morgan, and N. R. Landau. 1995. Human immunodeficiency virus type 1 viral protein R (Vpr) arrests cells in the G2 phase of the cell cycle by inhibiting p34cdc2 activity. J. Virol. 69:6705-6711

20. Herrmann, C. H., and A. P. Rice. 1993. Specific interaction of the human immunodeficiency virus Tat proteins with a cellular protein kinase. Virology 197:601-608

21. Herrmann, C. H., R. G. Carroll, P. Wei, K. A. Jones, and A. P. Rice. 1998. Tat-associated kinase, TAK, activity is regulated by distinct mechanisms in peripheral blood lymphocytes and promonocytic cell lines. J. Virol. 72:9881-9888

22. Johnson, D. G., and C. L. Walker. 1999. Cyclins and cell cycle checkpoints. Annu. Rev. Pharmacol. Toxicol. 39:295-312

23. Kashanchi, F., E. T. Agbottah, C. A. Pise-Masison, R. Mahieux, J. Duvall, A. Kumar, and J. N. Brady. 2000. Cell cycle-regulated transcription by the human immunodeficiency virus type 1 Tat transactivator. J. Virol. 74:652-660

24. Kashanchi, F., J. F. Duvall, and J. N. Brady. 1992. Electroporation of viral transactivator proteins into lymphocyte suspension cells. Nucleic Acids Res. 20:4673-4674

25. Lewis, P. F., and M. Emerman. 1994. Passage through mitosis is required for oncoretroviruses but not for the human immunodeficiency virus. J. Virol. 68:510-516

26. Lewis, P., M. Hensel, and M. Emerman. 1992. Human immunodeficiency virus infection of cells arrested in the cell cycle. EMBO J. 11:3053-3058

27. Li, G., M. Simm, M. J. Potash, and D. J. Volsky. 1993. Human immunodeficiency virus type 1 DNA synthesis, integration, and efficient viral replication in growth-arrested T cells. J. Virol. 67:3969-3977

28. Lori, F., A. Malykh, A. Cara, D. Sun, J. N. Weinstein, J. Lisziewicz, and R. C. Gallo. 1994. Hydroxyurea as an inhibitor of human immunodeficiency virus-type 1 replication. Science 266:801-805

29. Malley, S. D., J. M. Grange, F. Hamedi-Sangsari, and J. R. Vila. 1994. Synergistic anti-human immunodeficiency virus type 1 effect of hydroxamate compounds with 2′,3′-dideoxyinosine in infected resting human lymphocytes. Proc. Natl. Acad. Sci. USA 91:11017-11021

30. Malley, S. D., J. M. Grange, F. Hamedi-Sangsari, and J. R. Vila. 1994. Suppression of HIV production in resting lymphocytes by combining didanosine and hydroxamate compounds. Lancet 343:1292

31. Meijer, L., A. Borgne, O. Mulner, J. P. Chong, J. J. Blow, N. Inagaki, M. Inagaki, J. G. Delcros, and J. P. Moulinoux. 1997. Biochemical and cellular effects of roscovitine, a potent and selective inhibitor of the cyclin-dependent kinases cdc2, cdk2 and cdk5. Eur. J. Biochem. 243:527-536

32. Meijer, L., S. Leclerc, and M. Leost. 1999. Properties and potential-applications of chemical inhibitors of cyclin-dependent kinases. Pharmacol. Ther. 82:279-284

33. Nekhai, S., R. R. Shukla, and A. Kumar. 1997. A human primary T-lymphocyte-derived human immunodeficiency virus type 1 Tat-associated kinase phosphorylates the C-terminal domain of RNA polymerase II and induces CAK activity. J. Virol. 71:7436-7441

34. Okamoto, H., T. P. Cujec, B. M. Peterlin, and T. Okamoto. 2000. HIV-1 replication is inhibited by a pseudo-substrate peptide that blocks Tat transactivation. Virology 270:337-344

35. Pierson, T., J. McArthur, and R. F. Siliciano. 2000. Reservoirs for HIV-1: mechanisms for viral persistence in the presence of antiviral immune responses and antiretroviral therapy. Annu. Rev. Immunol. 18:665-708

36. Ross, E. K., A. J. Buckler-White, A. B. Rabson, G. Englund, and M. A. Martin. 1991. Contribution of NF-kappa B and Sp1 binding motifs to the replicative capacity of human immunodeficiency virus type 1: distinct patterns of viral growth are determined by T-cell types. J. Virol. 65:4350-4358

37. Schang, L. M., J. Phillips, and P. A. Schaffer. 1998. Requirement for cellular cyclin-dependent kinases in herpes simplex virus replication and transcription. J. Virol. 72:5626-5637

38. Schang, L. M., A. Rosenberg, and P. A. Schaffer. 1999. Transcription of herpes simplex virus immediate-early and early genes is inhibited by Roscovitine, an inhibitor specific for cellular cyclin-dependent kinases. J. Virol. 73:2161-2172

39. Schang, L. M., A. Rosenberg, and P. A. Schaffer. 2000. Roscovitine, a specific inhibitor of cellular cyclin-dependent kinases, inhibits herpes simplex virus DNA synthesis in the presence of viral early proteins. J. Virol. 74:2107-2120

40. Senderowicz, A. M., and E. A. Sausville. 2000. Roscovitine, a specific inhibitor of cellular cyclin-dependent kinases, inhibits herpes simplex virus DNA synthesis in the presence of viral early proteins. J. Natl. Cancer. Inst. 92:376-387

41. Siekevitz, M., S. F. Josephs, M. Dukovich, N. Peffer, F. Wong-Staal, and W. C. Greene. 1987. Activation of the HIV-1 LTR by T cell mitogens and the trans-activator protein of HTLV-I. Science. Science 238:1575-1578.

42. Spina, C. A., J. C. Guatelli, and D. D. Richman. 1995. Establishment of a stable, inducible form of human immunodeficiency virus type 1 DNA in quiescent CD4 lymphocytes in vitro. J. Virol. 69:2977-2988

43. Springett, G. M., R. C. Moen, S. Anderson, R. M. Blaese, and W. F. Anderson. 1989. Infection efficiency of T lymphocytes with amphotropic retroviral vectors is cell cycle dependent. J. Virol. 63:3865-3869

44. Tang, S., B. Patterson, and J. A. Levy. 1995. Highly purified quiescent human peripheral blood CD4⁺ T cells are infectible by human immunodeficiency virus but do not release virus after activation. J. Virol. 69:5659-5665

45. Trigon, S., H. Serizawa, J. W. Conaway, R. C. Conaway, S. P. Jackson, and M. Morange. 1998. Characterization of the residues phosphorylated in vitro by different C-terminal domain kinases. J. Biol. Chem. 273:6769-6775

46. Watkins, B. A., H. H. Dorn, W. B. Kelly, R. C. Armstrong, B. J. Potts, F. Michaels, C. V. Kufta, and M. Dubois-Dalcq. 1990. Specific tropism of HIV-1 for microglial cells in primary human brain cultures. Science 249:549-553

47. Wei, P., M. E. Garber, S. M. Fang, W. H. Fischer, and K. A. Jones. 1998. A novel CDK9-associated C-type cyclin interacts directly with HIV-1 Tat and mediates its high-affinity, loop-specific binding to TAR RNA. Cell 92:451-462

48. Weinberg, J. B., T. J. Matthews, B. R. Cullen, and M. H. Malim. 1991. Productive human immunodeficiency virus type 1 (HIV-1) infection of nonproliferating human monocytes. J. Exp. Med. 174:1477-1482

49. Zack, J. A., S. J. Arrigo, S. R. Weitsman, A. S. Go, A. Haislip, and I. S. Chen. 1990. HIV-1 entry into quiescent primary lymphocytes: molecular analysis reveals a labile, latent viral structure. Cell 61:213-222

50. Zhou, M., M. A. Halanski, M. F. Radonovich, F. Kashanchi, J. Peng, D. H. Price, and J. N. Brady. 2000. Tat modifies the activity of CDK9 to phosphorylate serine 5 of the RNA polymerase II carboxyl-terminal domain during human immunodeficiency virus type 1 transcription. Mol. Cell. Biol. 20:5077-5086

51. Zhu, Y., T. Pe'ery, J. Peng, Y. Ramanathan, N. Marshall, T. Marshall, B. Amendt, M. B. Mathews, and D. H. Price. 1997. Transcription elongation factor P-TEFb is required for HIV-1 tat transactivation in vitro. Genes Dev. 11:2622-2632. -hydroxypiperidin-1-yl.

1. Use of a compound of formula I 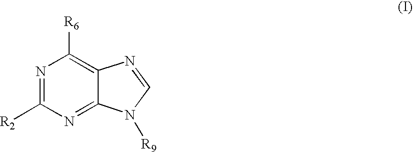
wherein
R₂ is R—NH wherein R is a branched or unbranched alkyl radical, a piperidinyl group or pyrrolidinyl group, each of which may be optionally substituted by one or more —OH, halogen, amino or hydroxyalkyl groups;
R₆ is phenylamino, benzylamino or pyridyl-methylamino, indan-5-amino, where in each case the aryl group may be unsubstituted or substituted by one or more —OR″, halogen, NO₂, amino or COOR′ groups, wherein R′ and R″ are each independently H or a branched or unbranched alkyl group; and
R₉ is a branched or unbranched alkyl group or a cycloalkyl group;
in the treatment of an autoimmune disorder.

2. Use according to claim 1 wherein R₂ is a branched or unbranched alkyl radical substituted by one or more OH groups.

3. Use according to claim 1 or claim 2 wherein R₂ is NHCH(R₄)CH(R₃)OH wherein R₃ and R₄ are each independently H or alkyl.

4. Use according to claim 3 wherein R₂ is selected from NH—CH(CHMe₂)CH₂OH, NHCH₂CH₂OH and NHCH(CH₂CH₃)CH₂OH.

5. Use according to claim 1 wherein R₂ is 2-hydroxymethylpyrrolidin-1-yl or 3-hydroxypiperidin-1-yl.

6. Use according to any preceding claim wherein R₆ is an unsubstituted benzylamino or phenylamino group.

7. Use according to any one of claims 1 to 5 wherein R₆ is a benzylamino or phenylamino group substituted by one or more chloro or COOR′ groups.

8. Use according to any one of claims 1 to 5 wherein R₆ is an unsubstituted benzylamino group or a phenylamino group substituted by one or more chloro or COOR′ groups.

9. Use according to claim 8 wherein R₆ is an unsubstituted benzylamino group or a 3-chlorophenylamino group or a 4-carboxy-3-chlorophenylamino group.

10. Use according to any one of claims 1 to 5 wherein R₆ is selected from pyridyl-2-yl-methylamino, pyridyl-3-yl-methylamino and pyridyl-4-yl-methylamino.

11. Use according to any preceding claim wherein R₉ is a branched or unbranched C₁₋₆ alkyl group.

12. Use according to claim 11 wherein R₉ is methyl or isopropyl.

13. Use according to claim 1 wherein the compound of formula I is

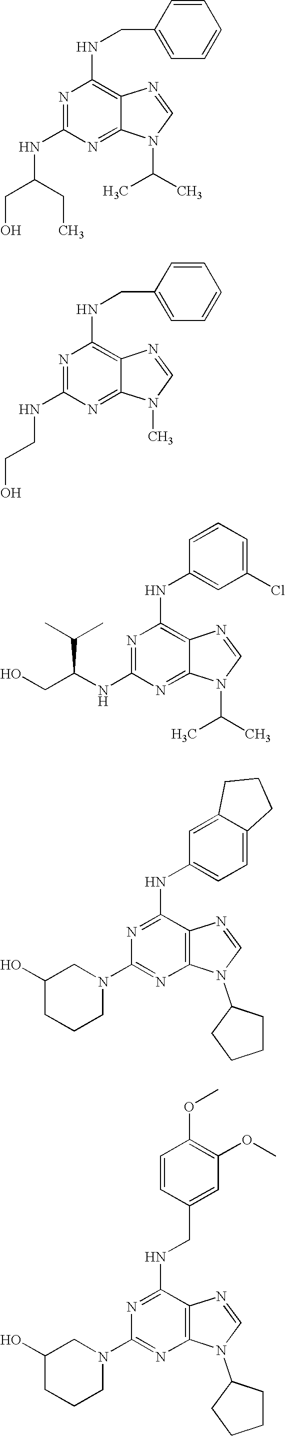

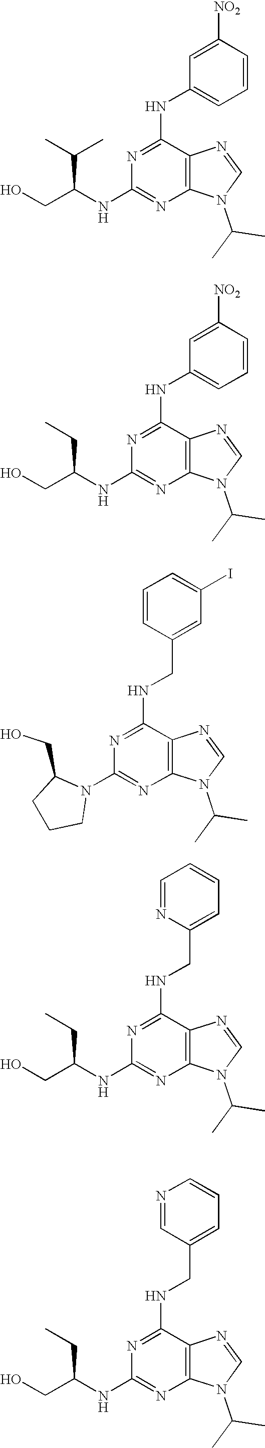

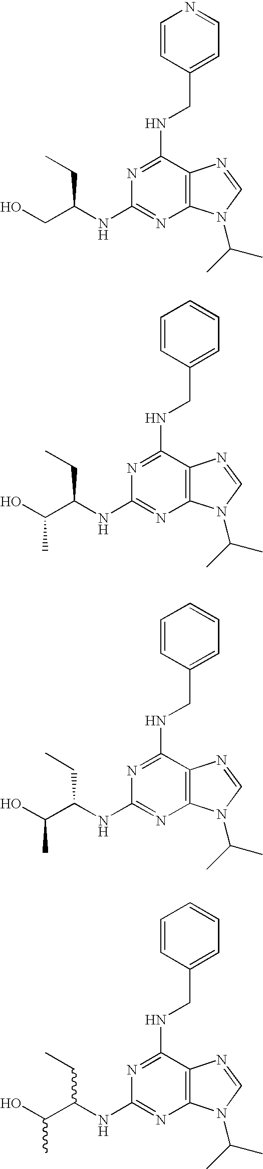

14. Use according to claim 13 wherein the compound of formula I is

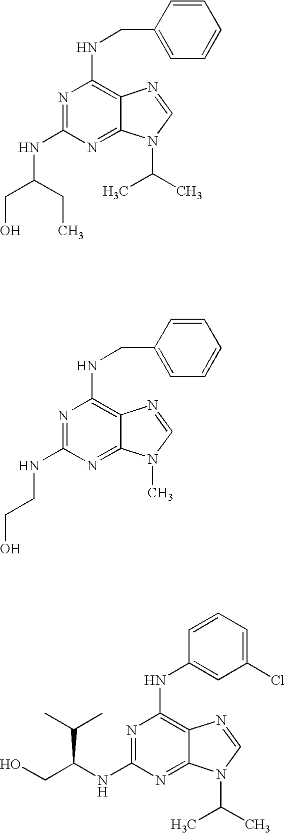

15. Use according to any preceding claim wherein the autoimmune disorder is a viral infection.

16. Use according to any preceding claim in the treatment of HIV-1-related disorders.

17. Use according to any preceding claim wherein the compound of formula I inhibits HIV-1 replication

18. Use according to claim 17 wherein the compound of formula I induces apoptosis in HIV-1 infected cells

19. Use according to any preceding claim wherein the compound of formula I inhibits Cyclin A- and/or E-associated histone HI kinase.

20. Use according to any preceding claim wherein the compound of formula I inhibits cdk7 and/or cdk9.

21. Use according to claim 1 in the treatment of AIDS.

22. A method of treating an autoimmune disorder comprising administering to a subject in need thereof a therapeutically effective amount of a compound of formula I 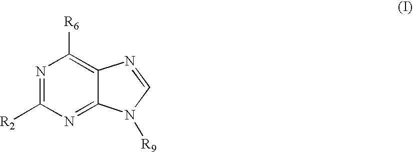
wherein
R₂ is R—NH wherein R is a branched or unbranched alkyl radical, a piperidinyl group or pyrrolidinyl group, each of which may be optionally substituted by one or more —OH, halogen, amino or hydroxyalkyl groups;
R₆ is phenylamino, benzylamino or pyridyl-methylamino, indan-5-amino, where in each case the aryl group may be unsubstituted or substituted by one or more —OR″, halogen, NO₂, amino or COOR′ groups, wherein R′ and R″ are each independently H or a branched or unbranched alkyl group; and
R₉ is a branched or unbranched alkyl group or a cycloalkyl group.

23. A method according to claim 22 wherein R₂ is a branched or unbranched alkyl radical substituted by one or more OH groups.

24. A method according to claim 22 or claim 23 wherein R₂ is NHCH(R₄)CH(R₃)OH wherein R₃ and R₄ are each independently H or alkyl.

25. A method according to claim 24 wherein R₂ is selected from NH—CH(CHMe₂)CH₂OH, NHCH₂CH₂OH and NHCH(CH₂CH₃)CH₂OH.

26. A method according to claim 22 wherein R₂ is 2-hydroxymethylpyrrolidin-1-yl or 3-hydroxypiperidin-1-yl.

27. A method according to any one of claims 22 to 26 wherein R₆ is an unsubstituted benzylamino group or a phenylamino group substituted by one or more chloro or COOR′ groups.

28. A method according to claim 27 wherein R₆ is an unsubstituted benzylamino group or a 3-chlorophenylamino group or a 4-carboxy-3-chlorophenylamino group.

29. A method according to any one of claims 22 to 26 wherein R₆ is selected from pyridyl-2-yl-methylamino and pyridyl-4-yl-methylamino.

30. A method according to claim 22 wherein R₉ is methyl or isopropyl.

31. A method according to claim 22 wherein the compound of formula I is

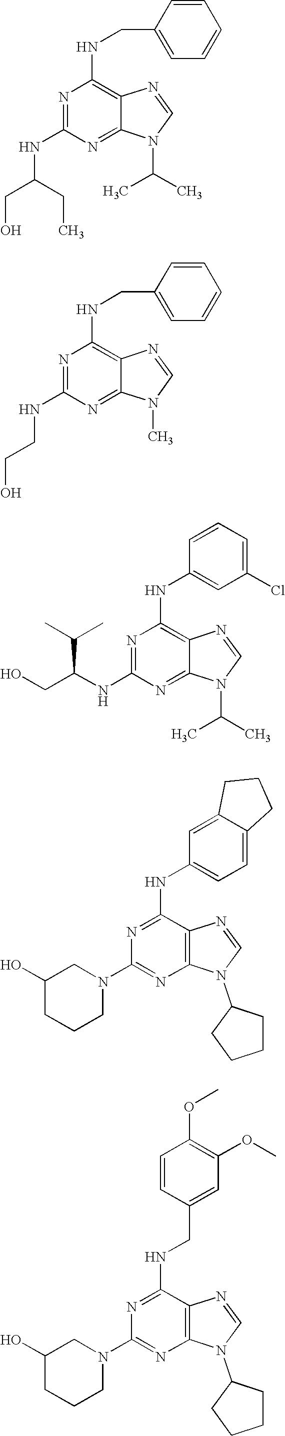

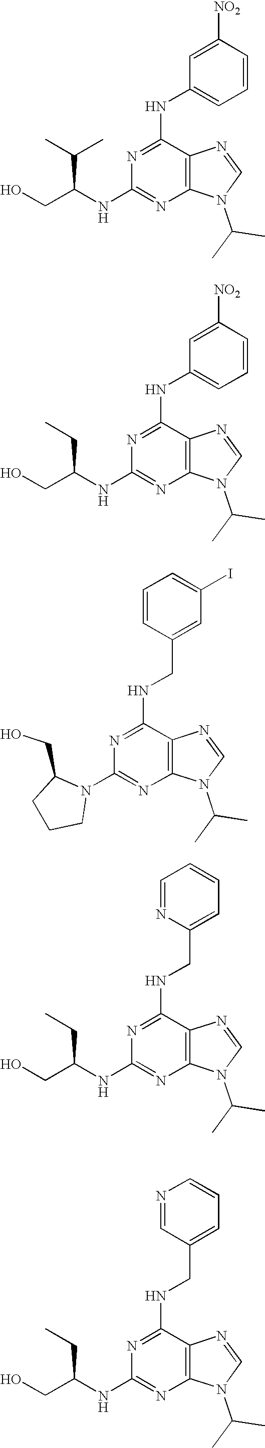

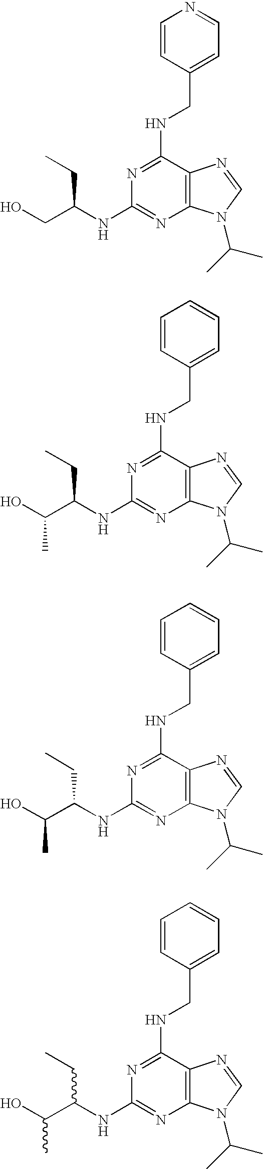

32. A method according to claim 31 wherein the compound of formula I is

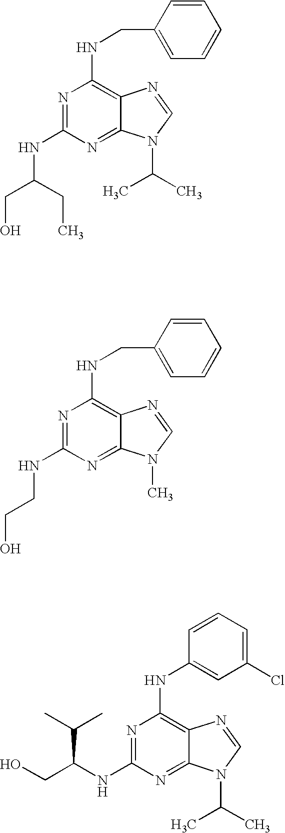

33. A method according to any one of claims 22 to 32 wherein the autoimmune disorder is HIV-1-related.
